(19) United States
(12) Patent Application Publication
(10) Pub. No.: US 2025/0041271 A1
(43) Pub. Date: Feb. 6, 2025

(54) ORAL SUSPENSION OF APREMILAST

(71) Applicant: AMGEN INC., Thousand Oaks, CA (US)

(72) Inventor: Yuchuan Gong, Thousand Oaks, CA (US)

(21) Appl. No.: 18/718,083

(22) PCT Filed: Dec. 23, 2021
(86) PCT No.: PCT/US21/65047

§ 371 Date: Jun. 10, 2024

(51) Int. Cl. A61K 31/4035; A61K 9/00; A61K 9/10; A61K 47/10; A61K 47/12; A61K 47/14; A61K 47/26; A61K 47/36; A61K 47/38

(52) U.S. Cl. CPC A61K 31/4035; A61K 9/0053; A61K 9/10; A61K 47/10; A61K 47/12; A61K 47/14; A61K 47/26; A61K 47/36; A61K 47/38

(57) ABSTRACT

Provided herein are suspensions for oral administration comprising a) apremilast, b) a suspending agent, c) a sweetener, d) one or more of a vehicle, a co-solvent, a buffering agent, a preservative, or a combination thereof. Also provided herein is a suspension for oral administration comprising a) apremilast, b) microcrystalline cellulose/carboxymethylcellulose sodium, c) xanthan gum, d) sucrose, e) sorbitol, f) glycerin, g) propylene glycol, h) citric acid monohydrate, i) methylparaben, j) propylparaben, and h) water.

### BACKGROUND

Apremilast is a phosphodiesterase subtype IV (“PDE4”) inhibitor and has been approved worldwide for the treatment of various diseases associated with inflammation. For example, in the United States apremilast is approved for the treatment of adult patients with moderate to severe plaque psoriasis who are candidates for phototherapy or system therapy, for the treatment of adult patients with active psoriatic arthritis, and for the treatment of adult patients with oral ulcers associated with Behcet's Disease. Apremilast was the first, and remains the only, orally administered PDE4 inhibitor approved for the treatment of these inflammatory diseases. The recommended dosage for adult patients with active PsA and psoriasis is 30 mg twice daily (BID) orally, following a 5-day titration that is intended to reduce the gastrointestinal (GI) symptoms associated with initial therapy. Apremilast remains under clinical development for the treatment of additional inflammatory autoimmune disorders that involve elevated inflammatory cytokine levels.

Apremilast is currently under investigation in the pediatric population for plaque psoriasis, psoriatic arthritis (e.g., Juvenile PsA), and Behcet's disease. To ensure patient compliance, however, a need exists for an apremilast formulation that is easy to swallow and administer and suitable for use in certain patient populations, such as pediatric patients. As such, apremilast suspension formulation is necessary to dose pediatric subjects unable to swallow an apremilast tablet.

In view of the foregoing, there remains a need for alternative oral dosage forms of apremilast that provide clinically desirable drug exposure.

### SUMMARY

The disclosure provides a suspension for oral administration comprising a) apremilast, b) a suspending agent, c) a sweetener, d) one or more of a vehicle, a co-solvent, a buffering agent, a preservative, or a combination thereof; and e) water.

The disclosure also provides a suspension for oral administration comprising a) apremilast, b) a blend of microcrystalline cellulose and carboxymethylcellulose sodium, c) xanthan gum, d) sucrose, e) sorbitol, f) glycerin, g) propylene glycol, h) citric acid monohydrate, i) methylparaben, j) propylparaben, and h) water.

### BRIEF DESCRIPTION OF THE FIGURES

FIG. 1 shows mean apremilast plasma concentration time profiles between oral suspensions and conventional tablets (Linear Scale).

FIG. 2 shows mean apremilast plasma concentration time profiles between oral suspensions and conventional tablets (Semi-logarithmic Scale).

FIG. 3 shows mean apremilast plasma concentration time profiles (food effect) oral suspension (Linear Scale).

FIG. 4 shows mean apremilast plasma concentration time profiles (food effect) oral suspension (Semi-logarithmic Scale).

FIG. 5A shows assay results from the stability studies of the disclosed suspensions at 25° C. and 60% relative humidity over 36 months.

FIG. 5B shows total degradation product results from the stability studies of the disclosed suspensions at 25° C. and 60% relative humidity over 36 months.

FIG. 5C shows methylparaben content results from the stability studies of the disclosed suspensions at 25° C. and 60% relative humidity over 36 months.

FIG. 5D shows propylparaben content results from the stability studies of the disclosed suspensions at 25° C. and 60% relative humidity over 36 months.

FIG. 5E shows viscosity results from the stability studies of the disclosed suspensions at 25° C. and 60% relative humidity over 36 months.

FIG. 5F shows density results from the stability studies of the disclosed suspensions at 25° C. and 60% relative humidity over 36 months.

FIG. 5G shows pH results from the stability studies of the disclosed suspensions at 25° C. and 60% relative humidity over 36 months.

FIG. 5H shows assay results from the stability studies of the disclosed suspensions at 40° C. and 75% relative humidity over 6 months.

FIG. 5I shows assay results from the degradation product results from the stability studies of the disclosed suspensions at 40° C. and 75% relative humidity over 6 months.

FIG. 5J shows methylparaben content results from the stability studies of the disclosed suspensions at 40° C. and 75% relative humidity over 6 months.

FIG. 5K shows propylparaben content results from the stability studies of the disclosed suspensions at 40° C. and 75% relative humidity over 6 months.

FIG. 5L shows viscosity results from the stability studies of the disclosed suspensions at 40° C. and 75% relative humidity over 6 months.

FIG. 5M shows density results from the stability studies of the disclosed suspensions at 40° C. and 75% relative humidity over 6 months.

FIG. 5N shows density results from the stability studies of the disclosed suspensions at 40° C. and 75% relative humidity over 6 months.

### DETAILED DESCRIPTION

Provided herein are suspensions for oral administration comprising a) apremilast, b) a suspending agent, c) a sweetener, d) one or more of a vehicle, a co-solvent, a buffering agent, a preservative, or a combination thereof; and e) water.

In some embodiments, the disclosure provides a suspension for oral administration comprising a) apremilast, b) microcrystalline cellulose/carboxymethylcellulose sodium, c) xanthan gum, d) sucrose, e) sorbitol, f) glycerin, g) propylene glycol, h) citric acid monohydrate, i) methylparaben, j) propylparaben, and h) water, as described herein. In some embodiments, the suspension comprises a) 5 mg/mL apremilast, b) 6.7 mg/mL microcrystalline cellulose/carboxymethylcellulose sodium, c) 1.8 mg/mL xanthan gum, d) 190 mg/mL sucrose, e) 190 mg/mL sorbitol, f) 133 mg/mL glycerin, g) 14 mg/mL propylene glycol, h) 1.8 mg/mL citric acid monohydrate, i) 1.5 mg/mL methylparaben, j) 0.17 mg/mL propylparaben, and h) water, as described herein.

The disclosed suspensions have a comparable (e.g., bioequivalent) AUC as an immediate-release oral apremilast tablet of comparable apremilast strength. In some embodiments, the suspensions have an area under the curve (AUC) of 80 to 125%, or 90% to 110%, of the AUC of an immediate-release oral apremilast tablet of comparable apremilast strength upon administration to a patient under fasting conditions.

### Apremilast

The disclosed suspensions comprise a suitable amount (e.g., concentration) of apremilast. Desirably, the suspensions contain an amount of apremilast that is suitable to provide a comparable area under the curve (AUC) (e.g., bioequivalent) as an immediate-release oral apremilast tablet of comparable apremilast strength. If the suspensions comprise too little apremilast, then the suspensions will not deliver a clinically suitable amount of apremilast and will not be efficacious when dosed. Conversely, if the suspensions comprise too much apremilast then the suspensions are inefficient in their use of apremilast, overly costly, and/or not optimally suited for the intended patient population (e.g., pediatric patients), and/or possibly induce adverse events. In some embodiments, apremilast is present in the suspensions in a total concentration of 3 to 10 mg/mL (e.g., 3.0, 3.5, 4.0, 4.5, 5.0, 5.5, 6.0, 6.5, 7.0, 7.5, 8.0, 8.5, 9.0, 9.5, or 10.0 mg/mL apremilast, as well as any range defined by any two of these values). In some embodiments, apremilast is present in the suspension in a total concentration of 5 mg/mL.

### Suspending Agent

The disclosed suspensions comprise a suspending agent. The suspending agent can be any suitable suspending agent. Some contemplated suspending agents include, but are not limited to microcrystalline cellulose, carboxymethylcellulose sodium, xanthan gum, hypromellose, polyvinylpyrrolidone (PVP), and commercially available suspending agents such as a blend of microcrystalline cellulose and carboxymethyl cellulose sodium such as Avicel® RC-591, or Avicel® CL-611. In some embodiments, the suspending agent comprises microcrystalline cellulose, carboxymethylcellulose sodium, methyl cellulose, ethyl cellulose, xanthan gum, xanthine gum, carrageenan; guar gum, locust bean gum, a carbomer, magnesium aluminum silicate (Veegum), or a combination thereof. In some embodiments, the suspending agent comprises a blend of microcrystalline cellulose and carboxymethylcellulose sodium blend and xanthan gum. In some embodiments, the suspending agent comprises xanthan gum.

The disclosed suspensions comprise a suitable amount of suspending agent. If the suspensions comprise too little suspending agent, then the suspensions may lack stability. If the suspension comprises too much suspending agent, then the suspensions may not have desirable physical properties (e.g., too viscous). In some embodiments, the suspending agent may be present in a total concentration of 5 mg/mL to 10 mg/mL (e.g., 5, 6, 7, 8, 9, or 10 mg/mL suspending agent, as well as any range defined by any two of these values). In some embodiments, the suspending agent is present in the suspension in a total concentration of 8.5 mg/mL.

In some embodiments, the suspensions comprise more than one suspending agent. In embodiments, wherein more than one suspending agent is present, each of the suspending agents can be present in the same or in different amounts. For example, in some embodiments wherein the suspending agent comprises a microcrystalline cellulose/carboxymethylcellulose sodium blend and xanthan gum, the microcrystalline cellulose/carboxymethylcellulose sodium blend and xanthan gum are present in a concentration ratio of 4 to 1 or less (e.g., 4:1, 3.9:1, 3.8:1, 3.7:1, 3.6:1, 3.5:1, 3.4:1, 3.3:1, 3.2:1, 3.1:1, or 3:1, as well as any range defined by any two of these values). By way of example, in some embodiments, the microcrystalline cellulose/carboxymethylcellulose sodium blend and xanthan gum are present in a concentration ratio of 3:4:1 to 4:1 (e.g., 3:5:1 to 3.9:1 or 3:6:1 to 3.8:1). In some embodiments, the microcrystalline cellulose/carboxymethylcellulose sodium blend and xanthan gum are present in a concentration ratio of 3.7:1. In some embodiments, the microcrystalline cellulose/carboxymethylcellulose sodium blend is present in the suspension at a total concentration of 6.7 mg/mL and the xanthan gum is present in the suspension at a total concentration 1.8 mg/mL.

### Sweetener

The disclosed suspensions comprise a sweetener. The sweetener can be any suitable sweetener. It is understood that the choice of sweetener, as well as any other component, is compatible with the desired patient population. For example, if a suspension is intended for a pediatric population, then a suitable component (e.g., sweetener) is used, which is compatible with the patient population. Desirably, the sweetener improves the palatability of the suspensions. Non-limiting examples of contemplated sweeteners include sucrose, dextrose, maltose, dextrins, trehalose, fructose, levulose, galactose, sorbitol, mannitol, xylitol, hydrogenated starch hydrolysates, maltitol, isomalt, erythritol, and lactitol. In some embodiments, the sweetener comprises sucrose, sorbitol, aspartame, fructose, lactose, or a combination thereof. In some embodiments, the sweetener comprises sucrose and sorbitol.

The disclosed suspensions comprise a suitable amount of sweetener. If the suspensions comprise too little sweetener, then the suspension may be bitter in taste and/or have a viscosity that is too low. Alternatively, or in addition, if the suspension comprises too much sweetener, then the suspension may not be palatable due to being overly sweet and/or have a viscosity that is too high. In some embodiments, the sweetener is present in the suspension in a total concentration of 500 mg/mL or less (e.g., 500, 490, 480, 470, 460, 450, 440, 430, 420, 410, 400, 390, 380, 370, 360, 350, 340, 330, 320, 310, 300, 290, 280, 270, 260, 250, 240, 230, 220, 210, or 200 mg/mL sweetener, as well as any range defined by any two of these values). In some embodiments, the sweetener is present in the suspension in a total concentration of 380 mg/mL.

In some embodiments, the suspensions comprise more than one sweetener. In these embodiments, each of the sweeteners is present in a suitable amount. For example, in some embodiments wherein the sweetener comprises sucrose and sorbitol, the sweetener comprises sucrose in a concentration of 150 to 225 mg/mL and comprises sorbitol in a concentration of 150 to 225 mg/mL. Nonlimiting concentrations for sucrose or sorbitol include, for example, 150, 155, 160, 165, 170, 175, 180, 185, 190, 195, 200, 205, 210, 215, 220, or 225 mg/mL, as well as any range defined by any two of these values. For example, in some embodiments the suspensions comprise 190 mg/mL sucrose and 190 mg/mL sorbitol (liquid), that is, a 1:1 concentration ratio of sucrose to sorbitol.

### Vehicle

In some embodiments, the disclosed suspensions comprise a vehicle. Desirably, the vehicle helps to stabilize the suspension and minimize settling of the particles. In some embodiments, the vehicle is used to decrease the hydration time of the suspending agents. In some embodiments the vehicle can be used to disperse the active ingredients. In some embodiments, the vehicle comprises glycerin.

The disclosed suspensions comprise a suitable amount of vehicle, when present. If the suspensions comprise too little vehicle, then the suspensions may not be stable. Alternatively, or in addition, if the suspension comprises too much vehicle, then the suspension may have unsuitable properties (e.g., too viscous). In some embodiments, the vehicle is present in the suspension in a total concentration of 100 mg/mL to 200 mg/mL (e.g., 100, 110, 120, 130, 140, 150, 160, 170, 180, 190, or 200 mg/mL vehicle, as well as any range defined by any two of these values). In some embodiments, the vehicle is present in the suspension in a total concentration of 133 mg/mL.

### Co-Solvent

In some embodiments, the disclosed suspensions comprise a co-solvent. Desirably, the co-solvent helps to improve solubility of the preservatives (e.g., methyl and propyl paraben) in the suspension and to improve stability. In some embodiments, the co-solvent comprises propylene glycol.

The disclosed suspensions comprise a suitable amount of co-solvent, when present. If the suspensions comprise too little, or too much, co-solvent, then the suspensions may lack desired physical properties and a shortened shelf-life. In some embodiments, the co-solvent is present in the suspension in a total concentration of 10 mg/mL to 20 mg/mL (e.g., 10, 11, 12, 13, 14, 15, 16, 17, 18, 19, or 20 mg/mL co-solvent, as well as any range defined by any two of these values). In some embodiments, the co-solvent is present in the suspension in a total concentration of 14 mg/mL.

In some embodiments, the co-solvent is heated to 40° C. to 50° C. to decrease the dissolution time.

### Buffering Agent

In some embodiments, the disclosed suspensions comprise a buffering agent. Non-limiting examples of contemplated buffering agents include acetic acid; ammonium carbonate; ammonium phosphate; boric acid; citric acid (e.g., citric acid anhydrous or citric acid monohydrate); lactic acid; phosphoric acid; citric acid salts (e.g., potassium citrate or sodium citrate); potassium metaphosphate; potassium phosphate dibasic; potassium phosphate monobasic; sodium acetate; sodium lactate; sodium phosphate dibasic; sodium phosphate monobasic; and succinic acid. In some embodiments, the buffering agent comprises citric acid (e.g., citric acid monohydrate) or salt thereof (e.g., potassium citrate or sodium citrate), or a combination thereof. In some embodiments, the buffering agent comprises citric acid or a hydrate thereof, a citric acid salt, tartaric acid, tartaric acid salt, ascorbic acid, ascorbic acid salt, phosphoric acid, phosphoric acid salt, acetic acid, acetic acid salt, or a combination thereof. In some embodiments, the buffering agent comprises citric acid monohydrate.

The disclosed suspensions comprise a suitable amount of buffering agent, when present. If the suspensions comprise too little buffering agent, then the suspensions may not have the desirable pH. Alternatively, or in addition, if the suspension comprises too much buffering agent, then the suspensions may lack stability. In some embodiments, the buffering agent is present in the suspension in a total concentration of 1 mg/mL to 5 mg/mL (e.g., 1.0, 1.1, 1.2, 1.3 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8. 3.9. 4.0, 4.1, 4.2, 4.3, 4.4, 4.5, 4.6, 4.7, 4.8, 4.9, or 5.0 mg/mL buffering agent, as well as any range defined by any two of these values). For example, in some embodiments, the buffering agent is present in the suspension in a total concentration of 1.6 mg/mL to 2 mg/mL. In some embodiments, the buffering agent is present in the suspension in a total concentration of 1.8 mg/mL.

### Preservative

In some embodiments, the disclosed suspensions comprise a preservative. Contemplated preservatives include, but are not limited to, anti-microbials, antioxidants, and agents that maintain stability and prevent microbial growth. Exemplary preservatives include ascorbic acid, ascorbyl palmitate, benzyl alcohol, xylitol, butylated hydroxyanisole (BHA), butylated hydroxytoluene (BHT), citric acid, erythorbic acid, fumaric acid, malic acid, propyl gallate, sodium ascorbate, sodium benzoate, sodium bisulfate, sodium metabisulfite, sodium sulfite, parabens (e.g., methyl-, ethyl-, butyl-), benzoic acid, potassium sorbate, and vanillin. In some embodiments, the preservative comprises methylparaben, propylparaben, or a combination thereof. In some embodiments, the preservative comprises methylparaben and propylparaben.

The disclosed suspensions comprise a suitable amount of preservative, when present. If the suspensions comprise too little preservative, the suspensions may not have microbial stability. Alternatively, or in addition, if the suspension comprises too much preservative, then the suspensions may be bitter in taste. In some embodiments, the preservative is present in the suspension in a total concentration of 0.5 mg/mL to 2.5 mg/mL (e.g., 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, or 2.5 mg/mL preservative, as well as any range defined by any two of these values). In some embodiments, the preservative is present is present in the suspension in a total concentration of 1 mg/mL to 2.5 mg/mL. In some embodiments, the preservative is present in the suspension in a total concentration of 1.7 mg/mL. In some embodiments, the preservative is present in the suspension in a total concentration of 1.5 mg/mL. In some embodiments, the preservative is present in the suspension in a total concentration of 1.2 mg/mL. In some embodiments, the preservative is present in the suspension in a total concentration of 1.1 mg/mL. In some embodiments, the preservative is present in the suspension in a total concentration of 0.9 mg/mL.

In some embodiments, the suspension comprises more than one preservative. In these embodiments, each of the preservatives is present in a suitable amount. For example, in some embodiments, the suspension comprises methylparaben and propylparaben. In some embodiments wherein the preservative comprises methylparaben and propylparaben, the concentration ratio of methylparaben to propylparaben is 9 to 1. In some embodiments comprising methylparaben and propylparaben, methylparaben is present in a concentration of 0.9 mg/mL or more and propylparaben is present in a concentration of 0.1 mg/mL or more. In some embodiments comprising methylparaben and propylparaben, methylparaben is present in a concentration of 0.9 mg/mL and propylparaben is present in a concentration of 0.1 mg/mL. In some embodiments comprising methylparaben and propylparaben, methylparaben is present in a concentration of 1.5 mg/mL and propylparaben is present in a concentration of 0.17 mg/mL. In some embodiments comprising methylparaben and propylparaben, methylparaben is present in a concentration of 1.1 mg/mL and propylparaben is present in a concentration of 0.12 mg/mL. In some embodiments comprising methylparaben and propylparaben, methylparaben is present in a concentration of 1.2 mg/mL and propylparaben is present in a concentration of 0.14 mg/mL.

### Water

The disclosed suspensions comprise water. The suspensions comprise a suitable amount of water to obtain the desired concentrations of components, as described herein, in the desired total volume of the suspension. In some embodiments, the suspensions comprise enough water to provide a suspension having a total volume of 1 mL. In some embodiments, the suspensions comprise enough water to provide a suspension having a total volume of 6 mL.

### Methods of Treating

In some embodiments, the disclosure provides methods of treating a patient suffering from a disease or disorder ameliorated by inhibiting PDE4 comprising administering to the patient the disclosed suspensions. In some embodiments, the patient is an adult patient. In some embodiments, the patient is a pediatric patient,

In some embodiments, disease or disorder is psoriasis, psoriatic arthritis, or Behcet's disease.

The disclosure provides methods of treating a patient suffering from a disease or disorder ameliorated by inhibiting PDE4 comprising administering to the patient the suspensions disclosed herein.

In some embodiments, the disease or disorder is asthma, arthritis, psoriasis, inflammation, chronic or acute obstructive pulmonary diseases, chronic or acute pulmonary inflammatory diseases, cutaneous lupus erythematosus, palmoplantar psoriasis, inflammatory bowel disease, Crohn's Disease, Behcet's Disease, or ulcerative colitis. In some embodiments, the disease or disorder arthritis. In some embodiments, the arthritis is psoriatic arthritis. In some embodiments, the disease or disorder is psoriasis. In some embodiments, the disease or disorder is ulcerative colitis. In some embodiments, the disease or disorder is Behcet's disease.

In some embodiments, the disclosed suspensions are administered after an initial titration with a twice-a-day apremilast formulation (e.g., a tablet formulation). In some embodiments, the method comprises administering only the disclosed suspensions. It should be understood that in embodiments wherein only the disclosed suspensions are administered, any initial titration(s) are conducted using the disclosed suspensions at a suitable dosage strength.

In some embodiments, the disclosed method comprises the following initial titration schedule: (i) suspension having 10 mg of apremilast in the morning on the first day of the administration; (ii) 10 mg in the morning and 10 mg after noon on the second day of administration; (iii) 10 mg in the morning and 20 mg after noon on the third day of administration; (iv) 20 mg in the morning and 20 mg after noon on the fourth day of administration; (v) 20 mg in the morning and 30 mg after noon on the fifth day of administration; and (vi) 30 mg apremilast twice daily Day 6 and every subsequent day of administration.

In some embodiments, the disclosed method comprises the following initial titration schedule: (i) 10 mg in the morning on the first day of administration; (ii) 10 mg in the morning and 10 mg after noon on the second day of administration; (iii) 10 mg in the morning and 20 mg after noon on the third day of administration; (iv) 20 mg in the morning and 20 mg after noon on the fourth day of administration; (v) 20 mg in the morning and 30 mg after noon on the fifth day of administration; and (vi) 30 mg in the morning and 30 mg after noon on Days 6-14 of administration; and a disclosed suspension having 30 mg apremilast twice daily Day 15 and every subsequent day of administration.

In some embodiments, the disclosed method comprises the following initial titration schedule (e.g., using a twice-a-day tablet formulation): (i) 10 mg in the morning on the first day of administration; (ii) 10 mg in the morning and 10 mg after noon on the second day of administration; (iii) 10 mg in the morning and 20 mg after noon on the third day of administration; (iv) 20 mg in the morning and 20 mg after noon on the fourth day of administration; (v) 20 mg in the morning and 30 mg after noon on the fifth day of administration; and (vi) an suspension as disclosed herein having 30 mg apremilast twice daily Day 6 and thereafter.

In some embodiments, the disclosed method comprises the following initial titration schedule: (i) 10 mg in the morning on the first day of administration; (ii) 10 mg in the morning and 10 mg after noon on the second day of administration; (iii) 10 mg in the morning and 20 mg after noon on the third day of administration; and (iv) an suspension as disclosed herein having 20 mg apremilast twice daily Day 4 and thereafter.

In some embodiments, the disclosed method comprises the following initial titration schedule: (i) 10 mg in the morning on the first day of administration; (ii) 10 mg in the morning and 10 mg after noon on the second day of administration; (iii) 10 mg in the morning and 20 mg after noon on the third day of administration; (iv) 20 mg in the morning and 20 mg after noon on Day 4 to Day 14; and (v) an suspension as disclosed herein having 20 mg apremilast twice daily Day 15 and thereafter.

In some embodiments, the disclosed method comprises the following initial titration schedule: (i) suspension having 5 mg of apremilast in the morning on the first day of the administration; (ii) 5 mg in the morning and 5 mg after noon on the second day of administration; (iii) 5 mg in the morning and 10 mg after noon on the third day of administration; (iv) 10 mg in the morning and 10 mg after noon on the fourth day of administration; (v) 10 mg in the morning and 20 mg after noon on the fifth day of administration; (vi) 20 mg twice daily Day 6 and every subsequent day of administration.

In some embodiments, the disclosure provides a packaged suspension comprising a suspension as disclosed herein, a bottle, and a stopper.

### EMBODIMENTS

1. A suspension for oral administration comprising:
a. apremilast;
b. a suspending agent;
c. a sweetener;
d. one or more of a vehicle, a co-solvent, a buffering agent, a preservative, or a combination thereof; and
e. water.

2. The suspension of embodiment 1, wherein the apremilast is present in the suspension in a total concentration of 3 to 10 mg/mL.

3. The suspension of embodiment 2, wherein the apremilast is present in the suspension in a total concentration of 5 mg/mL.

4. The suspension of any one of embodiments 1-3, wherein the suspending agent comprises microcrystalline cellulose, carboxymethylcellulose sodium, methyl cellulose, ethyl cellulose, xanthan gum, xanthine gum, carrageenan; guar gum, locust bean gum, a carbomer, magnesium aluminum silicate (Veegum), or a combination thereof.

5. The suspension of embodiment 4, wherein the suspending agent comprises a blend of microcrystalline cellulose and carboxymethyl cellulose sodium (microcrystalline cellulose/carboxymethyl cellulose sodium blend) and xanthan gum.

6. The suspension of embodiment 5, wherein the microcrystalline cellulose/carboxymethyl cellulose sodium blend and xanthan gum are present in a concentration ratio of 3.4:1 to 4:1 or less.

7. The suspension of embodiment 6, wherein the ratio is 3.7:1.

8. The suspension of any one of embodiments 1-7, wherein the suspending agent is present in the suspension in a total concentration of 8.5 mg/mL.

9. The suspension of any one of embodiments 1-8, wherein the sweetener comprises sucrose, sorbitol, aspartame, fructose, lactose, or a combination thereof.

10. The suspension of embodiment 9, wherein the sweetener comprises sucrose and sorbitol.

11. The suspension of embodiment 9 or 10, wherein the sweetener is present in the suspension in a total concentration of 500 mg/mL or less.

12. The suspension of embodiment 11, wherein the sweetener is present in the suspension in a total concentration of 380 mg/mL.

13. The suspension of any one of embodiments 1-9, wherein the sweetener comprises sucrose in a concentration of 150 to 225 mg/mL and sorbitol in a concentration of 150 to 225 mg/mL.

14. The suspension of embodiment 13, wherein the sweetener comprises sucrose and sorbitol in a concentration ratio of 1 to 1.

15. The suspension of any one of embodiments 1-14, wherein the suspension comprises a vehicle.

16. The suspension of embodiment 15, wherein the vehicle comprises glycerin.

17. The suspension of embodiment 15 or 16, wherein the vehicle is present in the suspension in a total concentration of 100 mg/mL to 200 mg/mL.

18. The suspension of embodiment 17, wherein the vehicle is present in a total concentration of 133 g/mL.

19. The suspension of any one of embodiments 1-18, wherein the suspension comprises a co-solvent.

20. The suspension of embodiment 19, wherein co-solvent comprises propylene glycol.

21. The suspension of embodiment 19 or 20, wherein the co-solvent is present in the suspension in a total concentration of 10 mg/mL to 20 mg/mL.

22. The suspension of embodiment 21, wherein the co-solvent is present in a total concentration of 14 mg/mL.

23. The suspension of any one of embodiments 1-22, wherein the suspension comprises a buffering agent.

24. The suspension of embodiment 23, wherein the buffering agent comprises citric acid or a hydrate thereof, a citric acid salt, tartaric acid, tartaric acid salt, ascorbic acid, ascorbic acid salt, phosphoric acid, phosphoric acid salt, acetic acid, acetic acid salt, or a combination thereof.

25. The suspension of embodiment 24, wherein the buffering agent comprises citric acid monohydrate.

26. The suspension of any one of embodiments 23-25, wherein the buffering agent is present in the suspension in a total concentration of 1 mg/mL to 5 mg/mL.

27. The suspension of embodiment 26, wherein the buffering agent is present in a total concentration of 1.8 mg/mL.

28. The suspension of any one of embodiments 1-27, wherein the suspension comprises a preservative.

29. The suspension of embodiment 28, wherein the preservative comprises methylparaben, propylparaben, or a combination thereof.

30. The suspension of embodiment 29, wherein the preservative comprises methylparaben and propylparaben.

31. The suspension of embodiment 30, wherein the methylparaben and propylparaben are present in a concentration ratio of 9 to 1.

32. The suspension of any one of embodiments 28-31, wherein the preservative is present in the suspension in a total concentration of 1 mg/mL to 2.5 mg/mL.

33. The suspension of embodiment 32, wherein the preservative is present in a total concentration of 1.7 mg/mL.

34. The suspension of any one of embodiments 30-32, wherein methylparaben is present in a concentration of 0.9 mg/mL or more and propylparaben is present in a concentration of 0.1 mg/mL or more.

35. A suspension for oral administration comprising a) apremilast, b) a blend of microcrystalline cellulose and carboxymethylcellulose sodium, c) xanthan gum, d) sucrose, e) sorbitol, f) glycerin, g) propylene glycol, h) citric acid monohydrate, i) methylparaben, j) propylparaben, and h) water.

36. The suspension of embodiment 35 comprising a) 5 mg/mL apremilast, b) 6.7 mg/mL of the blend of microcrystalline cellulose and carboxymethylcellulose sodium, c) 1.8 mg/mL xanthan gum, d) 190 mg/mL sucrose, e) 190 mg/mL sorbitol, f) 133 mg/mL glycerin, g) 14 mg/mL propylene glycol, h) 1.8 mg/mL citric acid monohydrate, i) 1.5 mg/mL methylparaben, j) 0.17 mg/mL propylparaben, and h) water.

37. The suspension of embodiment 35 comprising a) 5 mg/mL apremilast, b) 6.7 mg/mL of the blend of microcrystalline cellulose and carboxymethylcellulose sodium, c) 1.8 mg/mL xanthan gum, d) 190 mg/mL sucrose, e) 190 mg/mL sorbitol, f) 133 mg/mL glycerin, g) 14 mg/mL propylene glycol, h) 1.8 mg/mL citric acid monohydrate, i) 0.9 mg/mL methylparaben, j) 0.10 mg/mL propylparaben, and h) water

38. The suspension of embodiment 35 comprising a) 5 mg/mL apremilast, b) 6.7 mg/mL of the blend of microcrystalline cellulose and carboxymethylcellulose sodium, c) 1.8 mg/mL xanthan gum, d) 190 mg/mL sucrose, e) 190 mg/mL sorbitol, f) 133 mg/mL glycerin, g) 14 mg/mL propylene glycol, h) 1.8 mg/mL citric acid monohydrate, i) 1.1 mg/mL methylparaben, j) 0.12 mg/mL propylparaben, and h) water

39. The suspension of embodiment 35 comprising a) 5 mg/mL apremilast, b) 6.7 mg/mL of the blend of microcrystalline cellulose and carboxymethylcellulose sodium, c) 1.8 mg/mL xanthan gum, d) 190 mg/mL sucrose, e) 190 mg/mL sorbitol, f) 133 mg/mL glycerin, g) 14 mg/mL propylene glycol, h) 1.8 mg/mL citric acid monohydrate, i) 1.2 mg/mL methylparaben, j) 0.14 mg/mL propylparaben, and h) water

40. The suspension of any one of embodiments 1-39, having an AUC of 90% to 110% of the AUC of an immediate-release oral apremilast tablet of comparable apremilast strength upon administration to a patient under fasting conditions.

41. A method of treating a patient suffering from a disease or disorder ameliorated by inhibiting PDE4 comprising administering to the patient the suspension of any one of embodiments 1-40. 42. The method of embodiment 41, wherein the disease or disorder is psoriasis, psoriatic arthritis, or Behcet's disease.

43. The method of embodiment 41 or 42, wherein the patient is a pediatric patient.

44. A packaged suspension comprising the suspension of any one of embodiments 1-40, a bottle, and a stopper.

### EXAMPLES

The following examples further illustrate the disclosed methods of treatment, but of course, should not be construed as in any way limiting its scope.

The following abbreviations are used in the Examples: AUC_{0-∞} refers to area under the plasma concentration-time curve calculated from time zero to infinity; AUC₀₋ₜ refers to area under the concentration-time curve calculated from time zero to the last measured time point; CL/F refers to apparent clearance of drug from plasma after extravascular administration; Cₘₐₓ refers to observed maximum concentration; C_{trough} refers to observed plasma concentration at the end of the dosing interval; CI refers to confidence interval; CV % refers to coefficient of variation; t_{1/2} refers to terminal elimination half-life; t_{lag} refers to delay between time of administration and start of absorption lag time; Tₘₐₓ refers to time to Cₘₐₓ; Vz/F refers to apparent volume of distribution during the terminal phase; F refers to relative bioavailability of the oral suspension formulation compared to the tablet formulation;

Fᵣₑₗ₁ refers to relative bioavailability of each test formulation compared to the reference formulation; Fᵣₑₗ₂ refers to relative bioavailability of each dose-normalized test formulation compared to the dose-normalized reference formulation IR tablet.

### Example 1

This example illustrates a compounding process for preparing an oral suspension of apremilast in accordance with embodiments of the disclosure.

Sucrose (extra fine) and sorbitol liquid was added into purified water and mixed well to produce a sweetener solution. Microcrystalline cellulose/carboxymethylcellulose sodium and xanthan gum was added into the sweetener solution with mixing to obtain a homogeneous suspension. In a separate vessel, methylparaben and propylparaben in propylene glycol were dissolved. In some instances, the mixture of methylparaben and propylparaben can be heated (e.g., up to 60° C.) to facilitate dissolution. After dissolution, the solution was cooled to room temperature. The paraben solution was then added, along with citric acid monohydrate, to the previously prepared homogeneous suspension with mixing. The resulting suspension was then passed through an appropriate size mesh (e.g., 20 mesh). In a separate vessel, apremilast API was screened and dispersed into glycerin. The glycerin suspension was then mixed with the sieved suspension comprising the other components. Purified water was added (q.s.) to provide a bulk suspension having the desired volume and the bulk suspension was mixed.

The surge tank of the filling machine was filled with the bulk suspension with mixing before and during the fill process. Glass bottles were filled to the desired target weight (±5%). The necessary process controls (e.g., fill weight checks) were conducted. The filled glass bottles were fitted with caps.

### Example 2

This example illustrates the stability of the disclosed oral suspensions under various conditions and times, including accelerated stability testing conditions (e.g., 5° C., 25° C. at 60% RH, and 40° C. at 75% RH conditions) up to 36 months.

A suspension was prepared containing: 5 mg apremilast; 190 mg sucrose; 190 mg sorbitol; 133 mg glycerin; 6.7 mg MCC; 1.8 mg xanthan gum; 1.8 mg citric acid; 1.5 mg methylparaben, 0.172 mg propylparaben; 14 mg propylene glycol; and purified water qs to a volume of 1 mL. The suspension was packaged in amber glass bottles.

The suspension was evaluated using standard methods for the following properties: appearance; resuspendability; assay; methylparaben; propylparaben; degradation products; pH; microbial limits; container appearance; density; and viscosity.

The suspension consistently met acceptance criteria for stability at all time points through 1 month in when stored at 5° C. condition.

The suspension consistently met acceptance criteria for stability at all time points through 36 months in when stored at 25° C./60% RH condition. Signs of sedimentation were present starting at 6 months through 36 months, however the suspension resuspendability was within the specification of 10 seconds throughout the duration of the study regardless of the storage conditions. Moreover, the suspension consistently met acceptance criteria for stability at all time points through 6 months when stored at 40° C./75% RH condition.

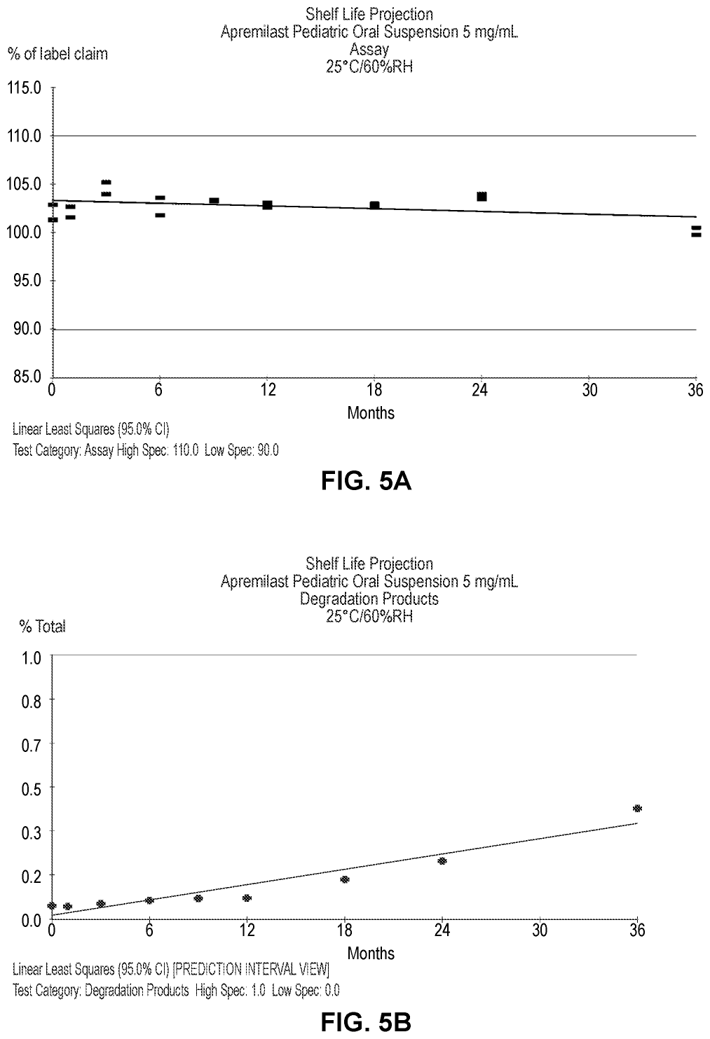

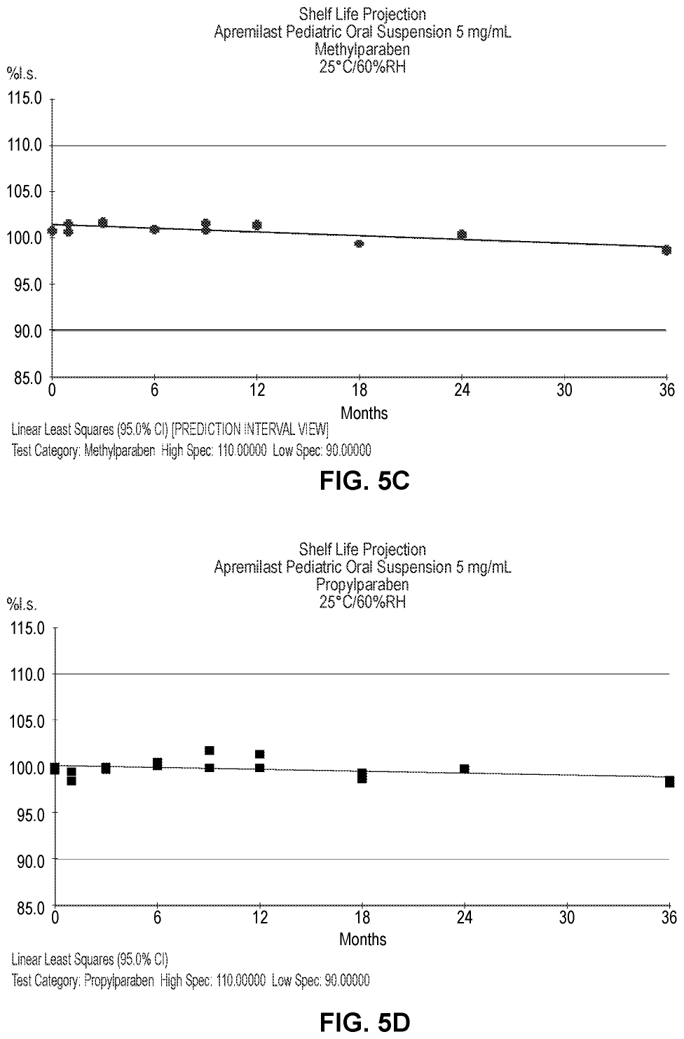

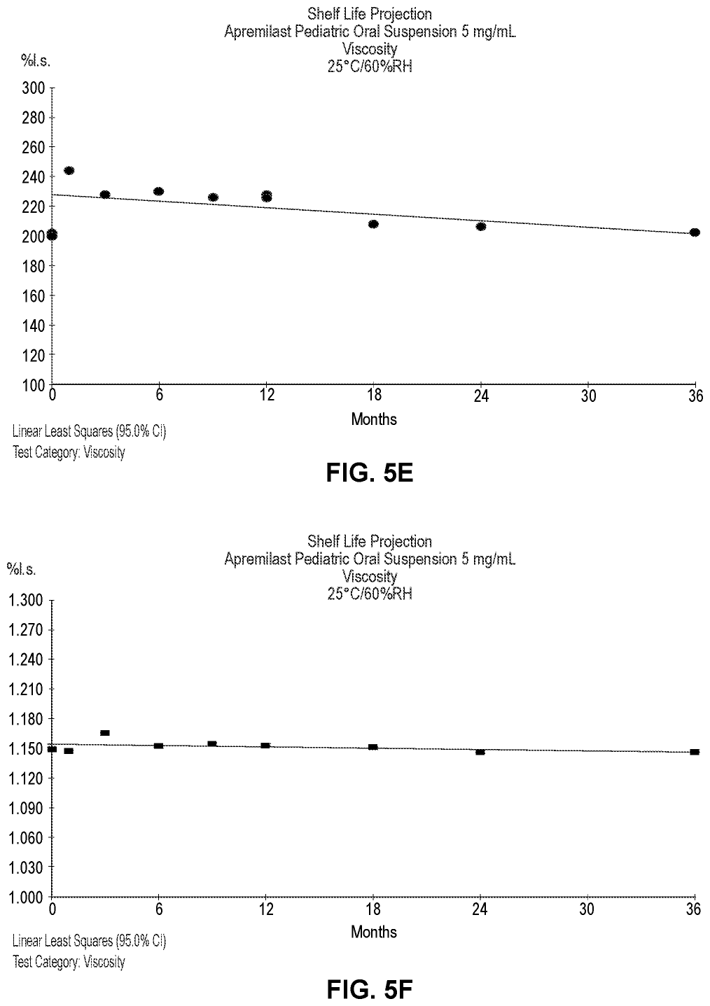

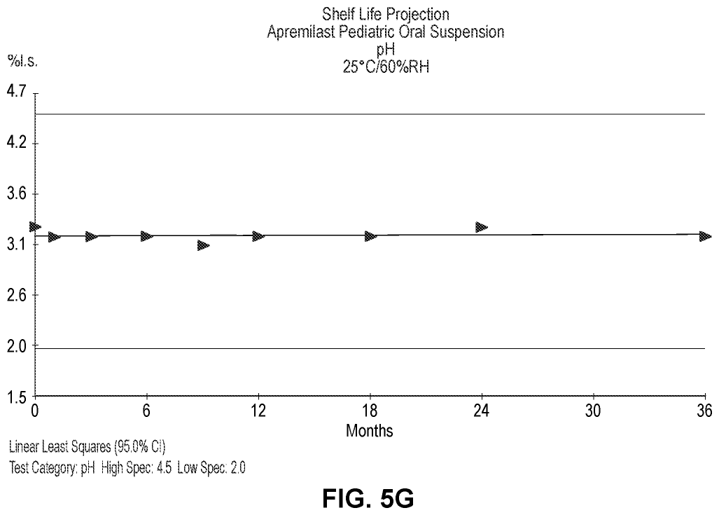

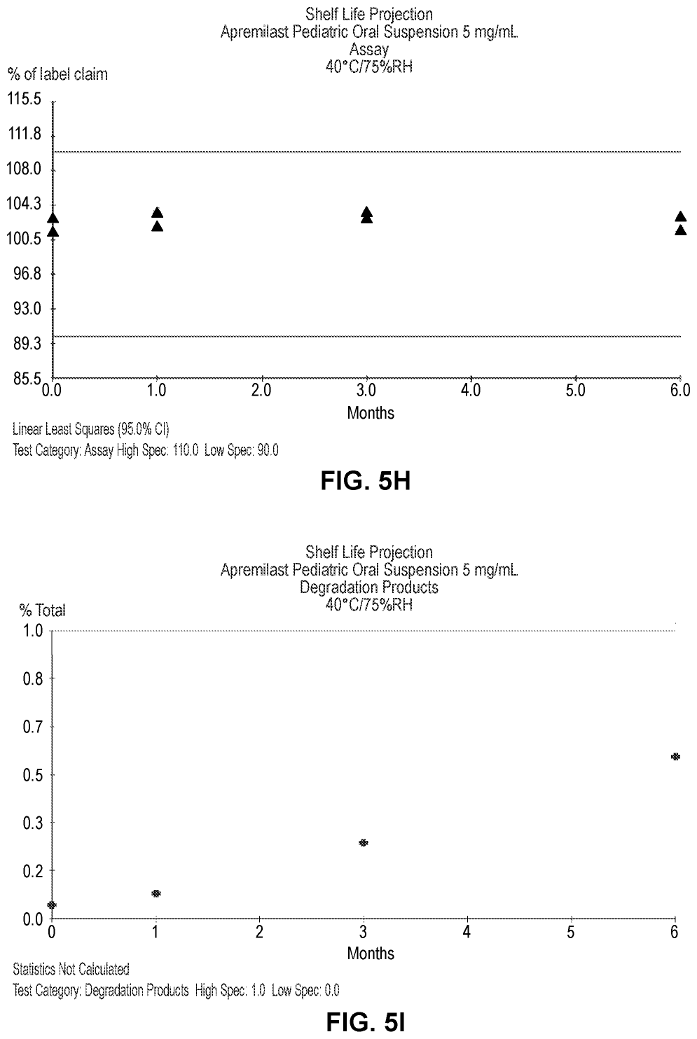

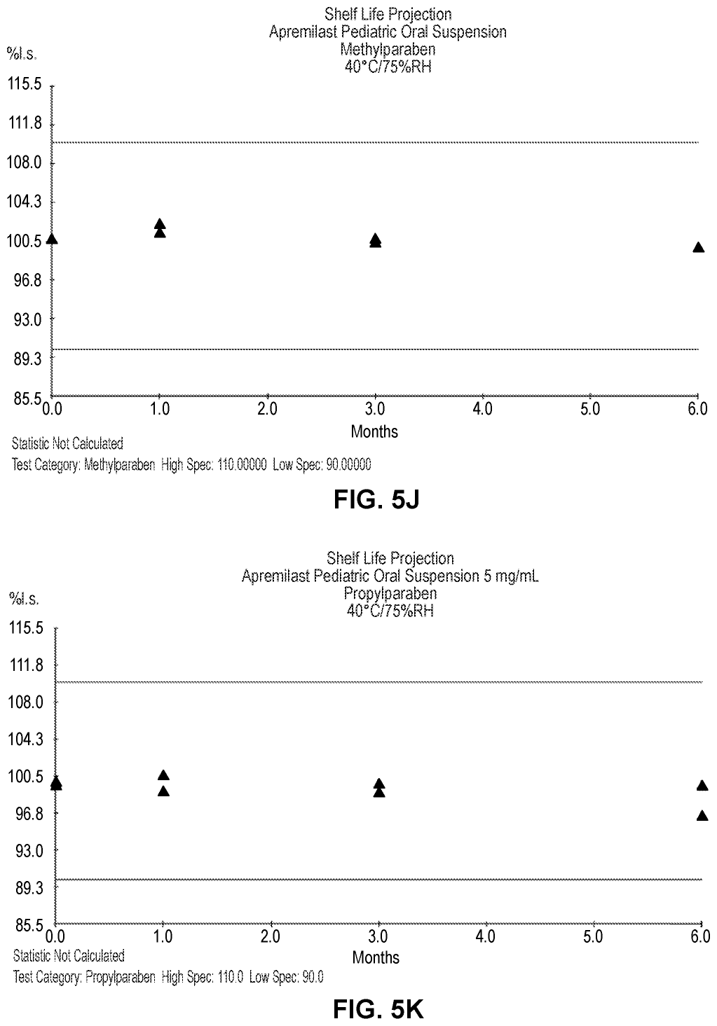

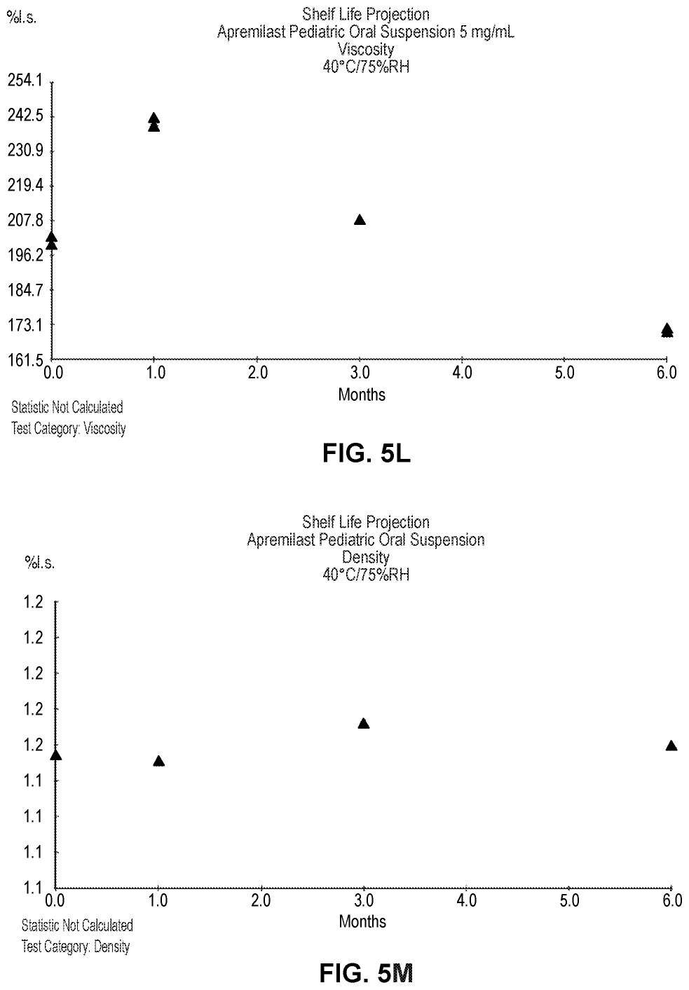

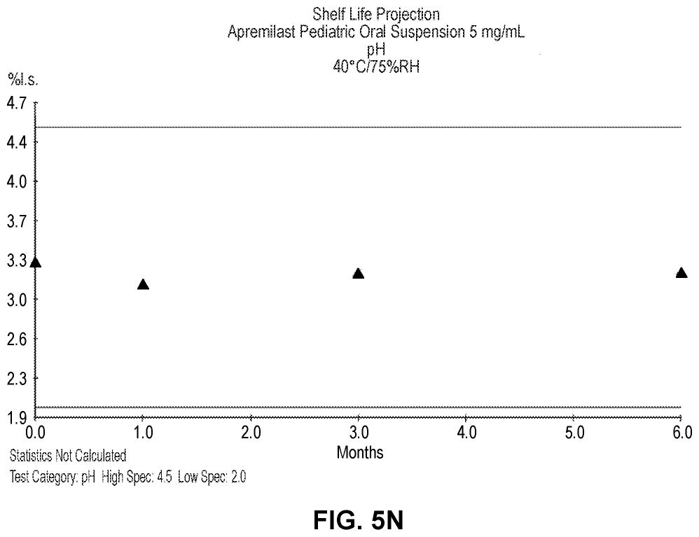

The results are summarized in FIGS. 5A-5G. The data depicted in FIGS. 5A-5G demonstrate that the disclosed suspensions have a shelf life of 36 months when stored at or below 25° C.

### Example 3

This example demonstrates the bioavailability of a single oral dose of 6 mL (30 mg) apremilast oral suspension relative to a 30 mg apremilast tablet in healthy subjects. This example also demonstrates the effect of food on the pharmacokinetics of the disclosed suspensions. The oral suspension comprised a) 5 mg/mL apremilast, b) 6.7 mg/mL of the blend of microcrystalline cellulose and carboxymethylcellulose sodium, c) 1.8 mg/mL xanthan gum, d) 190 mg/mL sucrose, e) 190 mg/mL sorbitol, f) 133 mg/mL glycerin, g) 14 mg/mL propylene glycol, h) 1.8 mg/mL citric acid monohydrate, i) 1.5 mg/mL methylparaben, j) 0.17 mg/mL propylparaben, and h) water. The 30 mg apremilast tablet comprised 30 mg apremilast; 78.75 mg microcrystalline cellulose (diluent); 180.0 mg lactose monohydrate (diluent); 9.0 mg croscarmellose sodium (disintegrant); 2.25 mg magnesium stearate (lubricant); 12.0 mg Opadry® II (film-coating).

This was an open-label, randomized, three-period, six-sequence crossover study in healthy subjects. The study consisted of a screening phase, baseline (Day −1), three treatment periods, and a follow-up phone call. Each treatment period was four days in duration (Day 1 through Day 4), with a five-day washout period between doses.

After the screening phase (Day −21 to Day −2), eligible subjects were admitted to the study center on Day −1 of Treatment Period 1 for baseline assessments. On Day 1 of Treatment Period 1, subjects were randomly assigned to one of the following six treatment sequences:

TABLE 1
Treatment Sequences

| Sequence | Period 1 | Period 2 | Period 3 |
|---|---|---|---|
| Sequence 1 (N = 5) | Treatment A | Treatment C | Treatment B |
| Sequence 2 (N = 5) | Treatment C | Treatment B | Treatment A |
| Sequence 3 (N = 6) | Treatment B | Treatment A | Treatment C |
| Sequence 4 (N = 6) | Treatment A | Treatment B | Treatment C |
| Sequence 5 (N = 6) | Treatment C | Treatment A | Treatment B |
| Sequence 6 (N = 6) | Treatment B | Treatment C | Treatment A |

On Day 1 of each treatment period, subjects received a single 30 mg oral dose of apremilast according to one of the following treatment sequences: Treatment A: Single oral dose of 30 mg apremilast tablet under fasted conditions; Treatment B: Single oral dose of 30 mg apremilast oral suspension formulation (6 mL) under fasted conditions; and Treatment C: Single oral dose of 30 mg apremilast oral suspension formulation (6 mL) under fed conditions. Subjects fasted overnight for at least 10 hours prior to dosing of Treatments A and B. No food or beverages were allowed for at least 4 hours after dosing.

For Treatment C, following an overnight fast of at least 10 hours, all subjects were served a high-fat meal. The standard high-fat breakfast was equal or equivalent to 2 eggs fried in butter, 2 strips of bacon, 2 slices of buttered toast or bread, 4 ounces of hash brown potatoes, and 8 ounces of whole milk. The entire meal was to be consumed within 20 to 25 minutes from the time the meal was served, and dosing occurred 30 minutes (±5 minutes) after the start of the meal. No food or beverages were allowed for at least 4 hours after dosing.

All treatments were administered with approximately 240 mL non-carbonated, room-temperature water. For all treatments, water was allowed as desired except for 1 hour before and 1 hour after drug administration (excluding water given with the IP). Lunch, dinner, and a snack were provided approximately 4, 9, and 13 hours post dose, respectively.

Each subject was asked to evaluate the aftertaste of the study drug post the administration of Treatments B and C, and prior to the administration of 240 mL non-carbonated, room-temperature water.

Subjects were confined to the study center from Day −1 of Treatment Period 1 through Day 4 of Treatment Period 3, thus including the five-day washout between doses. All subjects were discharged from the study center on Day 4 of Treatment Period 3 following completion of all required study procedures. A follow-up phone call to each subject was made approximately 4 days (±2 days) after discharge from the study center.

Blood samples for measurement of apremilast concentrations were collected at the following time points in each treatment period: predose (0 hour), and at 0.5, 1, 1.5, 2, 3, 5, 8, 12, 24, 36, 48, 60, and 72 hours post dose.

The following PK parameters were derived for apremilast by noncompartmental analysis. Actual sampling times were used in the calculation of PK parameters: AUC_{0-∞}, AUC₀₋ₜ, Cₘₐₓ, Tₘₐₓ, t_{1/2}, CL/F, V_{z}/F, F.

The software used in the data analysis and data presentation included Phoenix™ WinNonlin® Version 6.4 (Pharsight Corp., St. Louis, Missouri) and SAS Version 9.3 (SAS Institute Inc., Cary, North Carolina).

To assess the bioavailability between the apremilast oral suspension and tablet formulation and the effect of food on the PK of apremilast oral suspension formulation, an analysis of variance model, with treatment, sequence, and period as fixed effects and subject nested within sequence as a random effect, was performed on the natural log-transformed AUCs and Cₘₐₓ. The geometric means and percent ratios (Treatment B/Treatment A and Treatment C/Treatment B) of the geometric means are presented, along with the 95% confidence intervals (CIs) for the ratios.

For Tₘₐₓ, a non-parametric analysis was used to produce a median difference between treatments (Treatment B versus Treatment A and Treatment C versus Treatment B). The median, median difference, and 90% CI of the median difference were obtained from the Hodges-Lehmann estimate. The p-value was derived from the Wilcoxon signed-rank test.

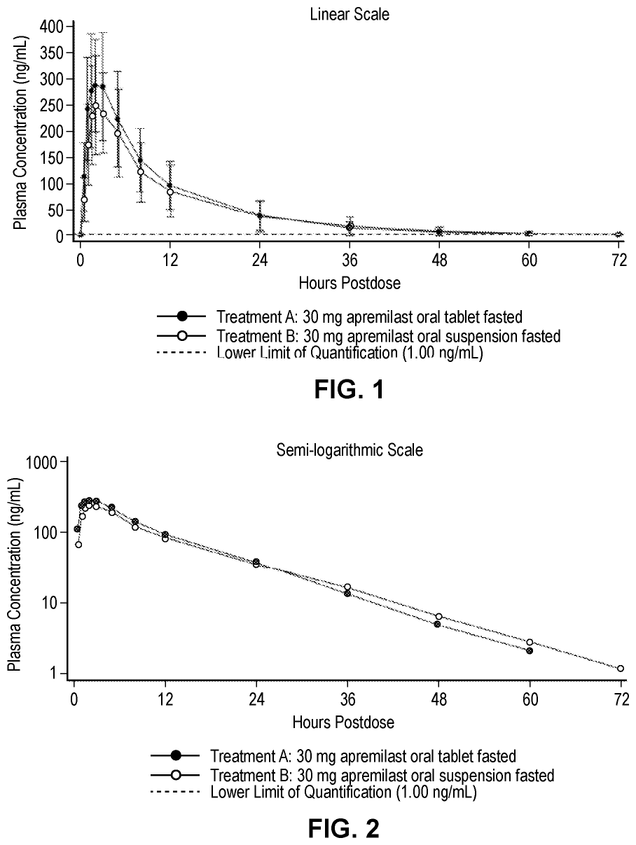

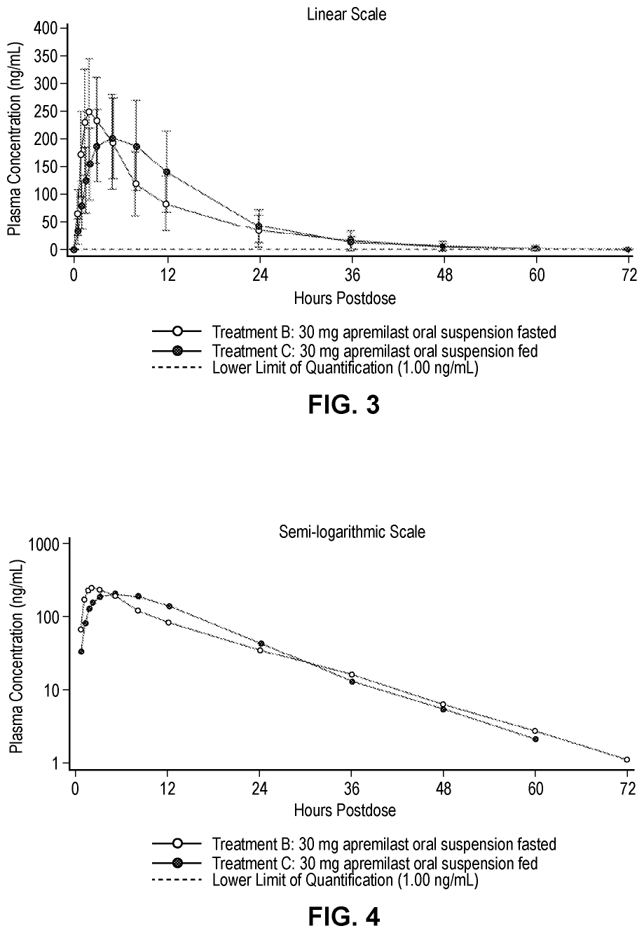

All subjects in treatment A (30 mg apremilast oral tablet fasted) had measurable apremilast plasma concentrations over the time up to hour 60 post dose (FIGS. 1 and 2) and in treatment B (30 mg apremilast oral suspension fasted) apremilast plasma concentrations over the entire sampling period from pre-dose to hour 72 post dose, and in treatment C (30 mg apremilast oral suspension fed), apremilast plasma concentrations over the entire sampling period from pre-dose to hour 60 post dose. (FIGS. 3 and 4).

All pharmacokinetic estimates were calculated using actual recorded blood drawing time. Plasma pharmacokinetic parameters are summarized in Table 2.

Under fed condition, the 30 mg oral suspension formulation achieved equivalent AUCs and comparable Cₘₐₓ to fasted condition. Although its Tₘₐₓ was delayed from median 2 hours to 5 hours post dose, this delay is not considered clinically meaningful.

TABLE 2
Geometric Mean (Geometric CV %) Estimates
of Apremilast Pharmacokinetic Parameters

| Treatment | 30 mg Tablet | | 30 mg Suspension | | 30 mg Suspension | |
|---|---|---|---|---|---|---|
| N of Subjects | fasted | | fasted | | fed | |
| AUC₀₋ₜ (ng · h/mL) | 3130 | (38.9) | 2740 | (43.1) | 3160 | (41.3) |
| AUC_{0-∞}(ng · h/mL) | 3160 | (38.7) | 2760 | (43.2) | 3190 | (41.3) |
| Cₘₐₓ (ng/mL) | 323 | (34.5) | 274 | (32.2) | 215 | (33.7) |
| Tₘₐₓ (hr)^{a} | 2.00 | (1.00, 5.00) | 2.00 | (1.50, 5.00) | 5.00 | (1.50, 12.00) |
| t_{1/2} (hr) | 7.89 | (34.5) | 8.51 | (31.8) | 7.54 | (30.8) |
| CL/F (L/h) | 9.51 | (38.7) | 10.9 | (43.2) | 9.42 | (41.3) |
| V_{z}/F (L) | 108 | (45.2) | 133 | (43.7) | 102 | (38.9) |
| F (%) | | | 87.6 | (16.1) | 101 | (14.3) |

The oral 30 mg (6 mL) suspension formulation achieved comparable AUCs and Cₘₐₓ, though about 13% to about 15% lower, compared to the 30 mg apremilast tablet (reference) following a single oral administration under fasting condition.

The results of the statistical analyses for AUC₀₋ₜ, AUC_{0-∞}, Cₘₐₓ and Tₘₐₓ for both the bioavailability of single oral dose of 6 mL (30 mg) apremilast oral suspension formulation (test) relative to the 30 mg apremilast tablet (reference) formulation and the effect of food on the apremilast oral suspension formulation are presented in Table 3. The analyses of median difference of Tₘₐₓ are presented in Table 4.

Under fasting condition, the bioavailability (AUC_{0-∞}) was equivalent between apremilast 6 mL (30 mg) oral suspension and apremilast 30 mg tablet since the 95% Confidence Intervals of the ratio of AUC_{0-∞} geometric mean shown in Table 3 was within the limits of 80% to 125%, i.e. the conventional bioequivalent criteria. The assessment of Cₘₐₓ was approximately 15% lower.

For the effect of food on the apremilast oral suspension formulation, the 95% Confidence Intervals of AUC_{0-∞} between fasting and fed, the ratio of geometric mean shown in Table 3 was also within the limits of 80% to 125%. Thus it achieved equivalent bioavailability. Under fed condition, Cₘₐₓ was lower relative to fasting condition.

The median Tₘₐₓ values are shown in Table 3. Under fasting condition, the treatment of 30 mg apremilast oral suspension (the test) had the median value comparable to the 30 mg apremilast tablet (the reference) with a difference of 0.25 hour. Median value of Tₘₐₓ of apremilast oral suspension fed was significantly delayed with median difference of 2.25 hour when compared to apremilast oral suspension fasted. The effect of food on Tₘₐₓ 90% CI of median difference comparison was statistically significant (p<0.0001).

TABLE 3
Statistical Comparison of Apremilast Pharmacokinetic Parameters Relating Exposure
After 30 mg Tablet Fasted or 30 mg Oral Suspension Fasted Administered

| | | | | | Ratio (%) | 90% CI of | |
|---|---|---|---|---|---|---|---|
| | | | | | of | Ratio of | Intra- |
| | | | | | Adjusted | Geometric | subject |
| | | | Geometric | | Geometric | Means | Variability |
| Parameter | Treatment | N | Mean | Comparison | Means | (%) | CV % |
| AUC₀₋ₜ | B | 34 | 2718.7 | B vs A | 87.6 | (83.0, 92.5) | 11.2 |
| (ng · h/mL) | C | 34 | 3133.4 | C vs B | 115.3 | (109.2, 121.7) | |
| | A | 34 | 3101.9 | | | | |
| AUC_{0-∞} | B | 34 | 2743.9 | B vs A | 87.8 | (83.2, 92.6) | 11.1 |
| (ng · h/mL) | C | 34 | 3155.3 | C vs B | 115.0 | (109.0, 121.7) | |
| | A | 34 | 3126.4 | | | | |
| Cₘₐₓ | B | 34 | 272.8 | B vs A | 84.9 | (77.3, 93.2) | 19.6 |
| (ng/mL) | C | 34 | 213.5 | C vs B | 78.2 | (71.2, 86.0) | |
| | A | 34 | 321.5 | | | | |

Treatment A: 30 mg apremilast tablet fasted.
Treatment B: 30 mg apremilast oral suspension fasted.
Treatment C: 30 mg apremilast oral suspension fed
geometric means, ratio (B/A or C/B) and 90% CI of the ratio of geometric means are from an ANOVA model with treatment, period, and sequence as a fixed effect, and subject nested within sequence as a random effect on the natural log transformed PK parameters.

TABLE 4
Statistical Comparison of Apremilast Pharmacokinetic Parameter
Tₘₐₓ After 30 mg Tablet Fasted and 30 mg Oral Suspension Administered

| | | | | | | 90% CI of | |
|---|---|---|---|---|---|---|---|
| | | | | | Median | Median | |
| Parameter | Treatment | N | Median | Comparison | Difference | Difference | p-value |
| Tₘₐₓ (h) | A | 34 | 2.00 | B vs A | 0.25 | (0.00, 0.50) | 0.1508 |
| | B | 34 | 2.50 | C vs B | 2.25 | (1.27, 3.25) | <0.0001 |
| | C | 34 | 5.00 | | | | |

Treatment A: 30 mg apremilast tablet fasted.
Treatment B: 30 mg apremilast oral suspension fasted.
Treatment C: 30 mg apremilast oral suspension fed
Note:
The median, median difference(B/A or C/B) and 90% CI of the median difference are from Hodges-Lehmann estimate. The P-value is from Wilcoxon signed-rank test.

Pharmacokinetics and Pharmacodynamics. Under the fasting condition, overall bioavailability (AUC_{0-∞}) were equivalent between the 6 mL (30 mg apremilast) oral suspension formulation and the 30 mg tablet in healthy subjects following a single oral dose, though Cₘₐₓ was approximately 15% lower. Under the fed condition, a single dose of 6 mL (30 mg apremilast) oral suspension formulation had equivalent bioavailability (AUC_{0-∞}), but lower Cₘₐₓ relative to the fasting condition. Median Tₘₐₓ was significantly delayed under the fed condition relative to the fasting condition, from hour 2 to hour 5 post dose. Overall, the results indicate that a single dose of 6 mL (30 mg apremilast) oral suspension had comparable bioavailability (AUC_{0-∞}) relative to 30 mg tablet under the fasting condition. The food does not have clinical meaningful effect on the PK exposure of apremilast following the oral administration of suspension formulation.

Under the fasting or fed conditions, the plasma concentration versus time profiles were similar between the 30 mg apremilast tablet and the 6 mL (30 mg) suspension formulation. Apremilast was rapidly absorbed with median Tₘₐₓ approximately 2 hours post dose. Terminal elimination half-life (t_{1/2}) for plasma apremilast was approximately 8 h, similar between the test and reference under the fasting or fed conditions. Overall bioavailability (AUC_{0-∞}) were equivalent between the 6 mL (30 mg apremilast) oral suspension formulation and the 30 mg tablet in healthy subjects following a single oral dose under fasting condition, though Cₘₐₓ was approximately 15% lower.

Under the fed condition, a single dose of 6 mL (30 mg apremilast) oral suspension formulation achieved equivalent bioavailability (AUC_{0-∞}), but lower in Cₘₐₓ compared to the fasting condition. Median Tmax was significantly delayed (by 2.5 hours) under the fed condition relative to the fasting condition. The results demonstrate that a single dose of 6 mL (30 mg apremilast) oral suspension formulation achieved equivalent bioavailability (AUC_{0-∞}), but lower Cₘₐₓ relative to the fasting condition.

Overall, the oral suspension formulation (30 mg apremilast) had pharmacokinetic exposure comparable to the 30 mg apremilast tablet. Therefore, the results suggest that the oral suspension formulation can be administered as an alternative formulation interchangeable with tablet formulations at the same nominal doses in clinical trials such as pediatric subjects who are not able to swallow the tablet or require low doses.

Apremilast was safe and well tolerated following administration of the apremilast oral suspension formulation under fasted and fed conditions and the apremilast tablet formulation under fasted conditions. The incidence of TEAEs was similar following the oral suspension (administered in the fasted and fed states) and the tablet formulation. The drug related TEAE with the highest incidence was diarrhea, followed by constipation, nausea, and headache. Diarrhea, nausea, and headache have also been the most frequently reported TEAEs in previous clinical pharmacology studies with apremilast.

Pharmacokinetics. Under the fasting condition, overall bioavailability (AUC_{0-∞}) were equivalent between the 6 mL (30 mg apremilast) oral suspension formulation and the 30 mg tablet in healthy subjects following a single oral dose, though Cₘₐₓ was approximately 15% lower. Under the fed condition, a single dose of 6 mL (30 mg apremilast) oral suspension formulation had equivalent bioavailability (AUC_{0-∞}), but lower Cₘₐₓ relative to the fasting condition. Median Tₘax was significantly delayed under the fed condition relative to the fasting condition, from hour 2 to hour 5 post dose. Overall, the results indicate that a single dose of 6 mL (30 mg apremilast) oral suspension had comparable bioavailability (AUC_{0-∞}) relative to 30 mg tablet under the fasting condition. The food does not have clinical meaningful effect on the PK exposure of apremilast following the oral administration of suspension formulation.

Safety. Single oral doses of 30 mg apremilast were safe and well tolerated by healthy subjects when administered as an oral suspension under fasted and fed conditions or as a tablet under fasted conditions.

### Example 4

This example demonstrates embodiments of the disclosed suspensions. Suspensions 4A, 4B, and 4C were prepared using the processes disclosed herein to provide batches of suspensions (26 L) containing different concentrations (60-80% level) of paraben preservatives, as shown in Table 5.

TABLE 5
Suspensions with Different Paraben Levels

| Component/ | 4A | 4B | 4C |
|---|---|---|---|
| Suspension¹ | (mg/mL) | (mg/mL) | (mg/mL) |
| Apremilast | 5.0 | 5.0 | 5.0 |
| Sucrose | 190 | 190 | 190 |
| Sorbitol (70%) | 190 | 190 | 190 |
| Glycerin | 133 | 133 | 133 |
| MCC/CCarboxymethyl- | 6.7 | 6.7 | 6.7 |
| ceullulose Na | | | |
| Xanthan Gum | 1.8 | 1.8 | 1.8 |
| Citric acid monohydrate | 1.8 | 1.8 | 1.8 |
| Methylparaben | 0.9 | 1.1 | 1.2 |
| Propylparaben | 0.1 | 0.12 | 0.14 |
| Propylene Glycol | 14 | 14 | 14 |
| Purified Water | QS to 1 mL | QS to 1 mL | QS to 1 mL |

¹batches were scaled and QS to 26 L

The foregoing examples are merely illustrative of embodiments of the disclosed processes described herein and are not intended to limit the disclosed methods. Variations and changes which are obvious to one skilled in the art are intended to be within the scope and nature of the disclosure which is defined in the appended claims.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing embodiments of the disclosure (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (for example, “such as”) provided herein, is intended merely to better illuminate embodiments of the disclosure and does not pose a limitation on the scope of the disclosure unless otherwise claimed. No language in the specification should be construed as indicating any non-claimed element as essential to the practice of the disclosure.

What is claimed:

1. A suspension for oral administration comprising:
a. apremilast;
b. a suspending agent;
c. a sweetener;
d. one or more of a vehicle, a co-solvent, a buffering agent, a preservative, or a combination thereof; and
e. water.

2. The suspension of claim 1, wherein the apremilast is present in the suspension in a total concentration of 3 to 10 mg/mL.

3. The suspension of claim 2, wherein the apremilast is present in the suspension in a total concentration of 5 mg/mL.

4. The suspension of any one of claims 1-3, wherein the suspending agent comprises microcrystalline cellulose, carboxymethylcellulose sodium, methyl cellulose, ethyl cellulose, xanthan gum, xanthine gum, carrageenan; guar gum, locust bean gum, a carbomer, magnesium aluminum silicate (Veegum), or a combination thereof.

5. The suspension of claim 4, wherein the suspending agent comprises a blend of microcrystalline cellulose and carboxymethyl cellulose sodium (microcrystalline cellulose/carboxymethyl cellulose sodium blend) and xanthan gum.

6. The suspension of claim 5, wherein the microcrystalline cellulose/carboxymethyl cellulose sodium blend and xanthan gum are present in a concentration ratio of 3.4:1 to 4:1 or less.

7. The suspension of claim 6, wherein the ratio is 3.7:1.

8. The suspension of any one of claims 1-7, wherein the suspending agent is present in the suspension in a total concentration of 8.5 mg/mL.

9. The suspension of any one of claims 1-8, wherein the sweetener comprises sucrose, sorbitol, aspartame, fructose, lactose, or a combination thereof.

10. The suspension of claim 9, wherein the sweetener comprises sucrose and sorbitol.

11. The suspension of claim 9 or 10, wherein the sweetener is present in the suspension in a total concentration of 500 mg/mL or less.

12. The suspension of claim 11, wherein the sweetener is present in the suspension in a total concentration of 380 mg/mL.

13. The suspension of any one of claims 1-9, wherein the sweetener comprises sucrose in a concentration of 150 to 225 mg/mL and sorbitol in a concentration of 150 to 225 mg/mL.

14. The suspension of claim 13, wherein the sweetener comprises sucrose and sorbitol in a concentration ratio of 1 to 1.

15. The suspension of any one of claims 1-14, wherein the suspension comprises a vehicle.

16. The suspension of claim 15, wherein the vehicle comprises glycerin.

17. The suspension of claim 15 or 16, wherein the vehicle is present in the suspension in a total concentration of 100 mg/mL to 200 mg/mL.

18. The suspension of claim 17, wherein the vehicle is present in a total concentration of 133 g/mL.

19. The suspension of any one of claims 1-18, wherein the suspension comprises a co-solvent.

20. The suspension of claim 19, wherein co-solvent comprises propylene glycol.

21. The suspension of claim 19 or 20, wherein the co-solvent is present in the suspension in a total concentration of 10 mg/mL to 20 mg/mL.

22. The suspension of claim 21, wherein the co-solvent is present in a total concentration of 14 mg/mL.

23. The suspension of any one of claims 1-22, wherein the suspension comprises a buffering agent.

24. The suspension of claim 23, wherein the buffering agent comprises citric acid or a hydrate thereof, a citric acid salt, tartaric acid, tartaric acid salt, ascorbic acid, ascorbic acid salt, phosphoric acid, phosphoric acid salt, acetic acid, acetic acid salt, or a combination thereof.

25. The suspension of claim 24, wherein the buffering agent comprises citric acid monohydrate.

26. The suspension of any one of claims 23-25, wherein the buffering agent is present in the suspension in a total concentration of 1 mg/mL to 5 mg/mL.

27. The suspension of claim 26, wherein the buffering agent is present in a total concentration of 1.8 mg/mL.

28. The suspension of any one of claims 1-27, wherein the suspension comprises a preservative.

29. The suspension of claim 28, wherein the preservative comprises methylparaben, propylparaben, or a combination thereof.

30. The suspension of claim 29, wherein the preservative comprises methylparaben and propylparaben.

31. The suspension of claim 30, wherein the methylparaben and propylparaben are present in a concentration ratio of 9 to 1.

32. The suspension of any one of claims 28-31, wherein the preservative is present in the suspension in a total concentration of 1 mg/mL to 2.5 mg/mL.

33. The suspension of claim 32, wherein the preservative is present in a total concentration of 1.7 mg/mL.

34. The suspension of any one of claims 30-32, wherein methylparaben is present in a concentration of 0.9 mg/mL or more and propylparaben is present in a concentration of 0.1 mg/mL or more.

35. A suspension for oral administration comprising a) apremilast, b) a blend of microcrystalline cellulose and carboxymethylcellulose sodium, c) xanthan gum, d) sucrose, e) sorbitol, f) glycerin, g) propylene glycol, h) citric acid monohydrate, i) methylparaben, j) propylparaben, and h) water.

36. The suspension of claim 35 comprising a) 5 mg/mL apremilast, b) 6.7 mg/mL of the blend of microcrystalline cellulose and carboxymethylcellulose sodium, c) 1.8 mg/mL xanthan gum, d) 190 mg/mL sucrose, e) 190 mg/mL sorbitol, f) 133 mg/mL glycerin, g) 14 mg/mL propylene glycol, h) 1.8 mg/mL citric acid monohydrate, i) 1.5 mg/mL methylparaben, j) 0.17 mg/mL propylparaben, and h) water.

37. The suspension of claim 35 comprising a) 5 mg/mL apremilast, b) 6.7 mg/mL of the blend of microcrystalline cellulose and carboxymethylcellulose sodium, c) 1.8 mg/mL xanthan gum, d) 190 mg/mL sucrose, e) 190 mg/mL sorbitol, f) 133 mg/mL glycerin, g) 14 mg/mL propylene glycol, h) 1.8 mg/mL citric acid monohydrate, i) 0.9 mg/mL methylparaben, j) 0.10 mg/mL propylparaben, and h) water

38. The suspension of claim 35 comprising a) 5 mg/mL apremilast, b) 6.7 mg/mL of the blend of microcrystalline cellulose and carboxymethylcellulose sodium, c) 1.8 mg/mL xanthan gum, d) 190 mg/mL sucrose, e) 190 mg/mL sorbitol, f) 133 mg/mL glycerin, g) 14 mg/mL propylene glycol, h) 1.8 mg/mL citric acid monohydrate, i) 1.1 mg/mL methylparaben, j) 0.12 mg/mL propylparaben, and h) water

39. The suspension of claim 35 comprising a) 5 mg/mL apremilast, b) 6.7 mg/mL of the blend of microcrystalline cellulose and carboxymethylcellulose sodium, c) 1.8 mg/mL xanthan gum, d) 190 mg/mL sucrose, e) 190 mg/mL sorbitol, f) 133 mg/mL glycerin, g) 14 mg/mL propylene glycol, h) 1.8 mg/mL citric acid monohydrate, i) 1.2 mg/mL methylparaben, j) 0.14 mg/mL propylparaben, and h) water

40. The suspension of any one of claims 1-39, having an AUC of 90% to 110% of the AUC of an immediate-release oral apremilast tablet of comparable apremilast strength upon administration to a patient under fasting conditions.

41. A method of treating a patient suffering from a disease or disorder ameliorated by inhibiting PDE4 comprising administering to the patient the suspension of any one of claims 1-40.

42. The method of claim 41, wherein the disease or disorder is psoriasis, psoriatic arthritis, or Behcet's disease.

43. The method of claim 41 or 42, wherein the patient is a pediatric patient.

44. A packaged suspension comprising the suspension of any one of claims 1-40, a bottle, and a stopper.
